(19) United States
(12) Patent Application Publication
(10) Pub. No.: US 2019/0240622 A1
(43) Pub. Date: Aug. 8, 2019

(54) SINGLE-USE PROCESS VESSEL WITH INTEGRATED FILTER MODULE

(71) Applicant: REPLIGEN CORPORATION, WALTHAM, MA (US)

(72) Inventor: Rudolf Pavlik, Waltham, MA (US)

(73) Assignee: REPLIGEN CORPORATION, WALTHAM, MA (US)

(21) Appl. No.: 16/263,466

(22) Filed: Jan. 31, 2019

Related U.S. Application Data
(62) Division of application No. 15/890,136, filed on Feb. 6, 2018.

(51) Int. Cl. B01D 61/18; B01D 69/08; F04B 43/04; B01D 61/22; C07K 1/34

(52) U.S. Cl. CPC B01D 61/18; B01D 69/08; C12N 7/02; B01D 61/22; C07K 1/34; F04B 43/04

(57) ABSTRACT

A single-use fluid storage and filtration system includes a process vessel, a filter module including a hollow fiber filter element, and a drive module coupled to the filter module. The filter module is fixed to the process vessel and is in fluid communication with the process vessel for filtering a fluid received from the process vessel. The drive module includes a pump to induce flow of the fluid between the filter module and the process vessel. The process vessel, filter module and drive module comprise a single integrated and sterilized assembly.

This application is a divisional of, and claims the benefit of priority to, U.S. patent application Ser. No. 15/890,136, filed Feb. 6, 2018, entitled “Single-Use Process Vessel with Integrated Filter Module,” which application is incorporated herein by reference in its entirety.

### BACKGROUND OF THE DISCLOSURE

### Field of the Disclosure

Embodiments of the disclosure relate generally to process filtration systems, and more particularly to a single-use process vessel, such as a bioreactor, having an integrated filter module.

### Discussion of Related Art

Filtration is typically performed to separate, clarify, modify, and/or concentrate a fluid solution, mixture, or suspension. In the biotechnology, pharmaceutical, and medical industries, filtration is vital for the successful production, processing, and analysis of drugs, diagnostics, and chemicals as well as many other products. As examples, filtration may be used to sterilize fluids and to clarify a complex suspension into a filtered “clear” fraction and an unfiltered fraction. Similarly, constituents in a suspension may be concentrated by removing or “filtering out” the suspending medium. Further, with appropriate selection of filter material, filter pore size and/or other filter variables, many other specialized uses have been developed. These uses may involve selective isolation of constituents from various sources, including cultures of microorganisms, blood, as well as other fluids that may be solutions, mixtures, or suspensions.

Biologics manufacturing processes have advanced through substantial process intensification. Both eukaryotic and microbial cell culture to produce recombinant proteins, virus-like particles (VLP), gene therapy particles, and vaccines now include cell growth techniques that can achieve 100e6 cells/ml or higher. This is achieved using cell retention devices that remove metabolic waste products and refresh the culture with additional nutrients. One of the most common means of cell retention is to perfuse a bioreactor culture using hollow fiber filtration using alternating tangential flow (ATF). Commercial and development scale processes use a device that controls a pump to perform ATF through a hollow fiber filter.

FIG. 1 illustrates a conventional arrangement of a process vessel 1 (which can be a bioreactor), a filter module 2 and a pump 4 for use in a biologics manufacturing process. The filter module 2 may include a filter element 6 disposed within a filter housing 8. The pump 4, which is illustrated as a diaphragm pump, is coupled to a bottom end 10 of the filter housing 8. Piping/tubing 12 is connected between a top end 14 of the filter housing 8 and the process vessel 1. Thus arranged, the pump 4 can act to move fluid back and forth between the process vessel 1 and the filter housing 8 so that the fluid can be filtered by the filter element 6. Filtered fluid can be drawn off via a fluid harvest port 18 disposed in the filter housing 8.

In some embodiments, the filter element 6 is a hollow-fiber module used to separate cells from spent media using ATF. Unlike systems that recirculate a culture through a filter in one direction, the ATF action constantly cleans the fibers of the filter element 6 with a periodic backflush action. With only a single connection to the process vessel 16, cells and media enter and leave the filter housing 8, flowing reversibly through the hollow fibers of the filter element 6. Flow is controlled by the pump 4, which generates a rapid low-shear flow between the process vessel 1 and the pump, ensuring rapid exchange and prompt return of cells to the process vessel and minimizing their residence outside the vessel. The choice of pore size for the hollow fibers determines what constituents are retained by, and which ones pass through, the filter element 6.

Conventionally, the process vessel 1, filter module 2 and pump 4 are separate components coupled together in the bioprocessing environment using mechanical couplings and piping/tubing 12 as shown. The pump 4 and filter module 2 are often encased in stainless steel and autoclaved prior to use to ensure sterility. The process vessel 1 and piping/tubing 12 may be separately sterilized.

As will be understood, the location of the filter module 2 in relation to the process vessel 1 is important to the proper functioning of the system, since improper installation can result in product damage or loss. For example, if the filter module 2 is placed too far from the process vessel 1, excess tubing length can undesirably impact the pumping and filtration efficiency of the system by introducing excessive dead space in the fluid path. Manufacturing space in the pharmaceutical industry is typically heavily populated with devices and equipment, which can make proper installation difficult.

In view of the foregoing, it would be desirable to provide a process vessel and filtration arrangement that simplifies installation of the system in a manner that reduces or eliminates errors that can impact process function and efficiency. It would also be desirable to provide a pre-sterilized process vessel and filtration arrangement that minimizes the total number of sterile connections that the user must make, and that minimizes or eliminates the need for the user to sterilize portions of the system.

### SUMMARY OF THE INVENTION

A single-use fluid storage and filtration system is disclosed. The system can include a process vessel and a filter module including a filter element. The filter module may be fixed to the process vessel. The filter module may be in fluid communication with the process vessel for filtering a fluid received from the process vessel. The system may also include a drive module coupled to the filter module. The drive module may include a pump to induce flow of the fluid between the filter module and the process vessel. The filter module may be disposed within the process vessel, or it may be disposed on a side surface of the process vessel. Alternatively, the filter module may be disposed directly beneath the process vessel. The system may further comprise supply and return tubing coupled between the filter module and the process vessel for moving the fluid between the filter module and the process vessel. The filter element may be a hollow fiber module. The process vessel may be a bioreactor and the filter element is a hollow fiber module for alternating tangential flow filtration.

A single-use fluid storage and filtration system is disclosed. The system may include a process vessel and a filter module including a filter element. The filter module may be fixed to the process vessel. The filter module may be in fluid communication with the process vessel for filtering a fluid received from the process vessel. A drive module may be coupled to the filter module, the drive module having a pump for inducing flow of the fluid between the filter module and the process vessel. The filter module may be disposed within the process vessel, or the filter module may be disposed on a side surface of the process vessel, or the filter module may be disposed directly beneath the process vessel. The system may further include supply and return tubing coupled between the filter module and the process vessel for moving the fluid between the filter module and the process vessel. The filter element may be a hollow fiber module. The process vessel may be a bioreactor and the filter element may be a hollow fiber module for alternating tangential flow filtration.

A single-use fluid storage and filtration system is disclosed. The system may include a bioreactor, and a filter module including a hollow fiber filter element, where the filter module is fixed to the bioreactor. The filter module may be in fluid communication with the bioreactor for filtering a fluid received from the bioreactor. A drive module may be coupled to the filter module. The drive module may include a pump for inducing flow of the fluid between the filter module and the bioreactor. The bioreactor, filter module and drive module may be a single integrated and sterilized assembly. The filter module may be disposed within the bioreactor and at least a portion of the drive module may be disposed outside of the bioreactor. The filter module may be disposed on a side surface of the bioreactor. Alternatively, the filter module may be disposed directly beneath the bioreactor. The system may further include supply and return tubing coupled between the filter module and the bioreactor for moving the fluid between the filter module and the bioreactor.

### BRIEF DESCRIPTION OF THE DRAWINGS

The accompanying drawings illustrate preferred embodiments of the disclosed method so far devised for the practical application of the principles thereof, and in which:

FIG. 1 is an isometric view of a conventional process vessel, filter and pump arrangement;

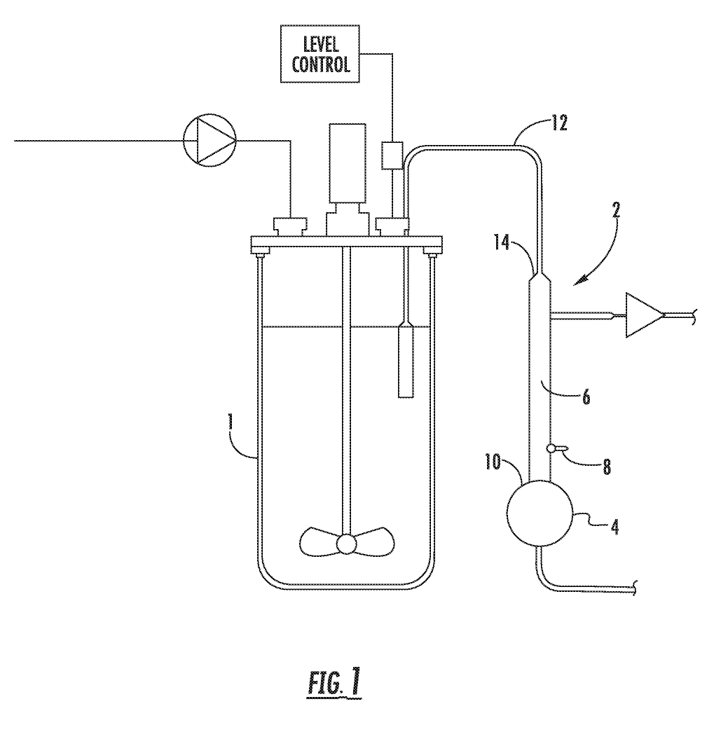

FIG. 2 is a schematic of a process vessel and filter module according to the present disclosure;

FIG. 3 is a schematic of an alternative process vessel and filter module according to the present disclosure;

FIG. 4 is a schematic of a further alternative process vessel and filter module according to the present disclosure;

FIG. 5 is a schematic of another alternative process vessel and filter module according to the present disclosure;

FIG. 6 is a schematic of a diaphragm pump arrangement for use with the process vessel and filter module according to the present disclosure; and

FIG. 7 is a schematic of a plunger pump arrangement for use with the process vessel and filter module according to the present disclosure.

### DESCRIPTION OF EMBODIMENTS

A process vessel having an integrated filter module and drive module is disclosed. In some embodiments, the process vessel, filter module and drive module are sterilized as a unit during manufacture, and are delivered to the user in a pre-sterilized form. The filter module can be coupled to the drive module for moving fluid from the process vessel in alternating directions through the filter module. The arrangement can be employed for conducting a rapid, low sheer, Alternating Tangential Flow (ATF) of fluid through the filter module, which in some embodiments includes a hollow fiber filter (HFF) element. Such a system has applications in perfusion of cultured animal cells as well as other varied filtration applications.

In some embodiments, a single-use process vessel, such as a bioreactor, is combined with a single-use filter module, such as a hollow fiber module, and an associated drive system (referred to herein as a “drive module”) to result in a single integrated and sterilized assembly. The integrated and sterilized assembly is then provided to the user for installation as a unit. The integrated and sterilized assembly can be a single-use assembly, which can be disposed of after a particular filtration evolution is complete. As will be described in greater detail later, the assembly can be provided in one of several configurations, depending on the application.

In some embodiments, the single-use system comprises a flexible process vessel (i.e., a closed system “bag”) with the single-use filter module and filter element may be either disposed inside the bag, or attached to a side of the bag. The bag can be inflated, and all connections can be made through appropriate sealing ports of the bag. External connectivity can be through septic connectors, thus forming an entire fluid filtration, sampling and harvesting loop. In other embodiments, the process vessel may be a rigid “tank”-like vessel. As will be described in greater detail later, coupling between the filter module and the process vessel (or between the drive module and the process vessel for embodiments in which the filter module is disposed interior to the process vessel) would be via septic connectivity, and a connection may be made through the process vessel to a flexible flange. In this way, connectivity of the components of the system can be universal, regardless of the filter module location.

It will be appreciated that although the filter module and the drive module will be referred to throughout the description as separate elements, such a convention is for ease of description, and the filer module and drive module can also constitute a single module. That is, the drive module may, in part or in its entirety, be a part of the filter module. Thus, in some embodiments the filter module and the drive module may be separately manufactured and coupled together, while in other embodiments the drive module may be integrally formed with the filter module. In yet further embodiments, the filter module may include a portion of the elements of the drive module (e.g., an upper housing and/or a diaphragm), while the drive module includes the remaining components (e.g., lower housing and/or diaphragm or plunger).

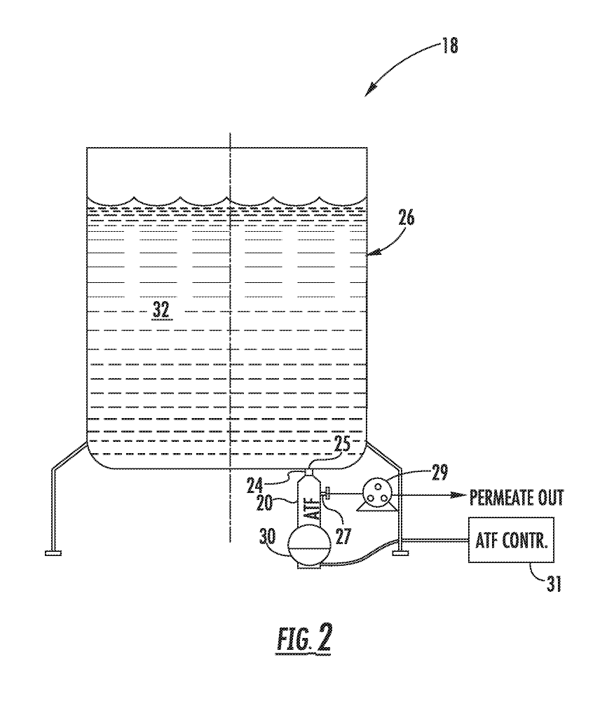

Referring now to FIGS. 2-5, various arrangements of a single-use process vessel and filter system 18 are shown. The system 18 can include a filter module 20 and a drive module 30 positioned in a selected relationship with respect to the process vessel 26. The location of the filter module 20 and drive module 30 in relation to the process vessel 26 can selected based on the size of the process vessel and the specifics of the application, as will be discussed in greater detail below. As will be appreciated, adjusting the position of the filter module 20 with respect to the process vessel 26 can provide more robust and clearly defined cell culture movement between the filter module and process vessel.

It will be appreciated that although the illustrated embodiments show the process vessel 26 as a rigid “tank,” that the process vessel can alternatively be a flexible process vessel (i.e., a closed system “bag”), or any other appropriate vessel configuration. It will also be appreciated that although each of the filter module 20 placement options will be described in relation to a single filter module/element, single or multiple-filter configurations can be employed in any or all of the arrangements. Where multiple filters are used, some or all the filters may be activated at any given time. Multiple filter activation may be employed if additional square footage of filtration media is required. Single filter activation in a multi-filter arrangement may be used as a backup.

FIG. 2 shows an embodiment of a single-use process vessel and filter system 18 in which the filter module 20 and filter element (not shown) are positioned beneath (i.e., external to) the process vessel 26. An upper end 24 of the filter module is directly coupled to an aseptic connector 25 of the process vessel 26. As such, with this embodiment the normal supply and return tubing used to fluidly couple the filter module 20 to the process vessel 26 is eliminated. As will be appreciated, providing the filter module 20 under the process vessel 26 may be beneficial for smaller filter sizes and where the process fluid 32 is a viscous liquid.

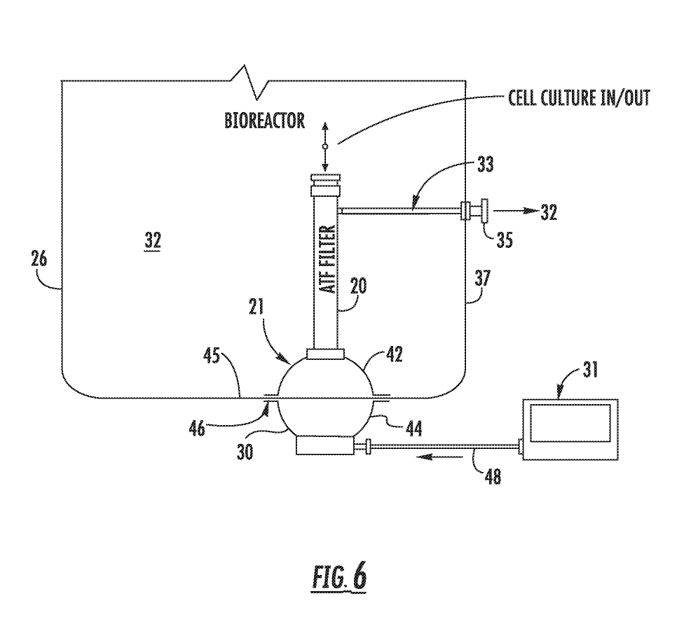

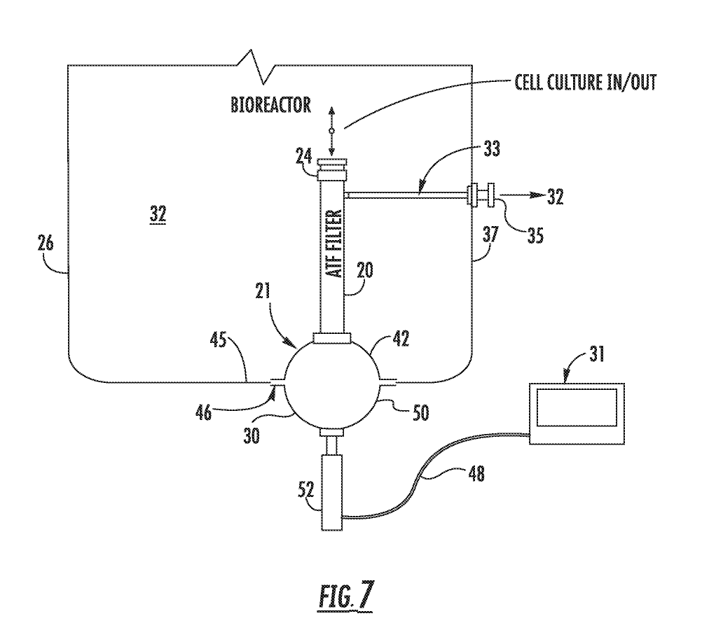

The drive module 30 is coupled beneath the filter module 20 and can comprise a pump (see FIGS. 6 and 7) coupled to an ATF controller 31 for inducing ATF of fluid 32 from the process vessel 26 through the filter element (not shown) within the filter module. Where the pump is a diaphragm pump, the connection between the pump and the ATF controller 31 may comprise supply and exhaust gas lines. Where the pump is a plunger pump, the connection between the pump and the ATF controller 31 may comprise an electrical or control connection for activating a linear actuator or servo coupled to the pump.

The filter module 20 is also provided with a fluid harvest port 27 which is coupled to an appropriate pump 29 for drawing permeate from the filter module at a desired rate and volume.

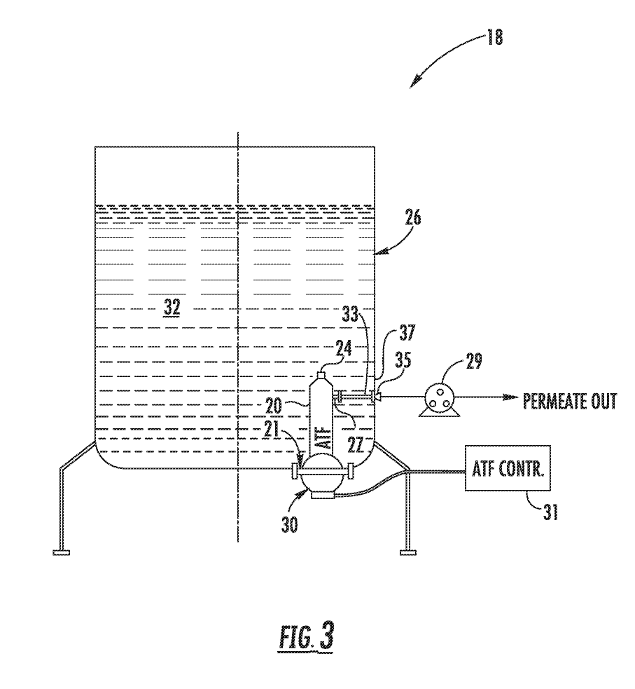

FIG. 3 shows an embodiment of a single-use process vessel and filter system 18 in which the filter module 20 and filter element (not shown) are positioned within the process vessel 26 such that the filter module is submerged in the process liquid during use. In the illustrated embodiment, a lower end 21 of the filter module 20 is directly coupled to the process vessel 26. With this embodiment, the normal supply and return tubing used to fluidly couple the filter module 20 to the process vessel 26 is eliminated. Rather, process fluid within the process vessel 26 is drawn into, and expelled from, the filter module 20 through an opening in the upper end 24 of the filter module. Providing the filter module 20 inside the process vessel 26 optimizes liquid exchange through the filter element since there is no need for a connecting tube between the filter module and the process vessel. Additionally, with this embodiment there will be no exposure of cells outside of the protected environment that the process vessel 26 provides.

The drive module 30 is coupled below the filter module 20, and is positioned directly beneath the process vessel 26. In some embodiments, the drive module 30 may be coupled directly to the bottom of the process vessel 26. Alternatively, the drive module 30 may be supported by the filter module 20 without directly connecting to the process vessel. The drive module 30 can comprise a pump (see FIGS. 6 and 7) coupled to an ATF controller 31 for inducing ATF of fluid 32 from the process vessel 26 through the filter element (not shown) within the filter module 20. Where the pump is a diaphragm pump, the connection between the pump and the ATF controller 31 may comprise supply and exhaust gas lines. Where the pump is a plunger pump, the connection between the pump and the ATF controller 31 may comprise an electrical or control connection for activating a linear actuator or servo coupled to the pump.

The filter module 20 can be provided with a fluid harvest port 27 which is coupled, via internal piping or tubing 33 to an aseptic coupling 35 disposed in or on the side 37 (or other surface) of the process vessel 26. An appropriate pump 29 can be coupled to the aseptic coupling 35 for drawing permeate from the filter module 20 at a desired rate and volume.

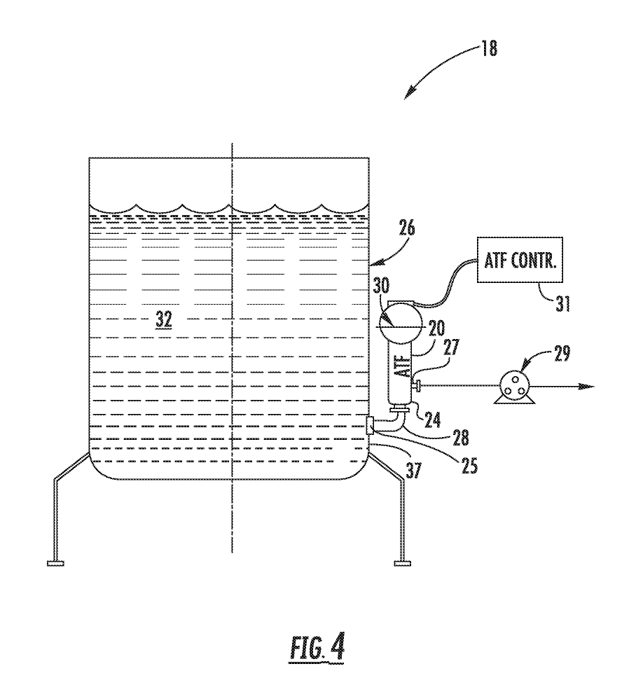

FIG. 4 shows an embodiment single-use process vessel and filter system 18 in which the filter module 20 and filter element (not shown) are mounted to a side of (i.e., external to) the process vessel 26. Supply and return tubing 28 is coupled between a bottom end 24 of the filter module 20 and an aseptic connector 25 mounted to the side 37 (or other surface) of the process vessel 26. Positioning the filter module 20 on a side of process vessel 26 and upside down can be advantageous, for example, for micro carrier applications with a minimal length of supply and return tubing 28 between the filter module and the process vessel 26.

The drive module 30 is coupled above the filter module 20 and is positioned to couple to an actuator (not shown) for inducing ATF of fluid 32 from the process vessel 26 through the filter element. The drive module 30 can comprise a pump (see FIGS. 6 and 7) coupled to an ATF controller 31 for inducing ATF of fluid 32 from the process vessel 26 through the filter element (not shown) within the filter module 20. Where the pump is a diaphragm pump, the connection between the pump and the ATF controller 31 may comprise supply and exhaust gas lines. Where the pump is a plunger pump, the connection between the pump and the ATF controller 31 may comprise an electrical or control connection for activating a linear actuator or servo coupled to the pump. The filter module 20 can be provided with a fluid harvest port 27 which is coupled to an appropriate pump 29 for drawing permeate from the filter module 20 at a desired rate and volume.

It will be appreciated that although the illustrated embodiment shows the drive module 30 positioned above the filter module 20, and the supply and return tubing 28 is coupled to a bottom end 24 of the filter module 20, that the orientation could also be reversed, with the drive module positioned below the filter module and the supply and return tubing coupled to a top end of the filter module.

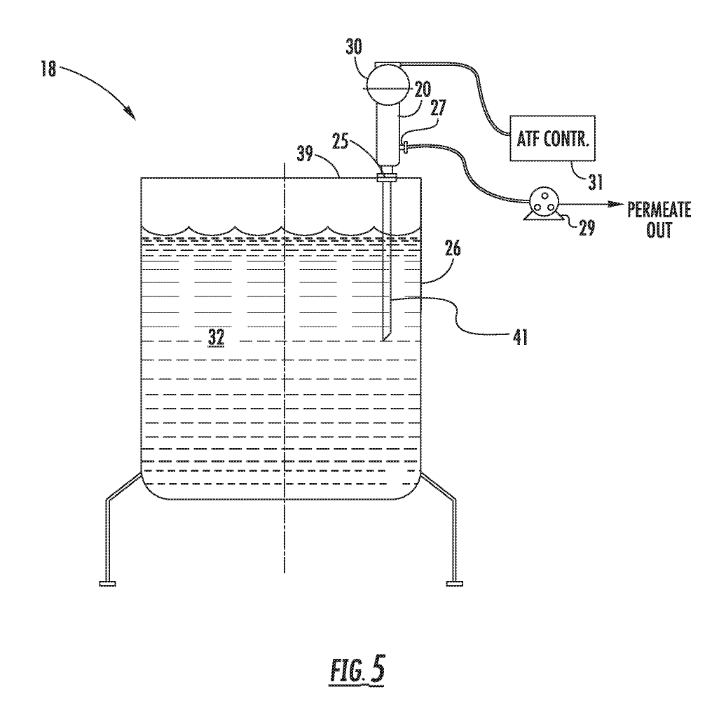

FIG. 5 shows an embodiment of a single-use process vessel and filter system 18 in which the filter module 20 and filter element (not shown) are positioned on top, or otherwise above, (i.e., external to) the process vessel 26. A lower end 24 of the filter module 20 is directly coupled to an aseptic connector 25 disposed on an upper portion 39 of the process vessel 26. A dip tube 41 or other appropriate fluid conduit extends from the aseptic connector 25 to a location within the process vessel 26 below the surface of the process fluid 32. As will be appreciated, positioning the filter module 20 on the top of the process vessel 26 can conserve space, for applications in which the system 18 is installed in confined quarters.

The drive module 30 is coupled above the filter module 20 and can comprise a pump (see FIGS. 6 and 7) coupled to an ATF controller 31 for inducing ATF of fluid 32 from the process vessel 26 through the filter element (not shown) within the filter module. Where the pump is a diaphragm pump, the connection between the pump and the ATF controller 31 may comprise supply and exhaust gas lines. Where the pump is a plunger pump, the connection between the pump and the ATF controller 31 may comprise an electrical or control connection for activating a linear actuator or servo coupled to the pump. The filter module 20 is also provided with a fluid harvest port 27 which is coupled to an appropriate pump 29 for drawing permeate from the filter module at a desired rate and volume.

As will be appreciated, each of the illustrated embodiments provides convenience of placement and connectivity between the filter module 20 and the process vessel 26. Since the filter module 20 is positioned in or on the process vessel 26, the length of the supply/return tubing 28 is minimized or eliminated, which thus minimizes the dead volume between the module and the vessel. Since the displacement of the pump must be greater than the dead volume in the supply/return tubing, the disclosed arrangements can advantageously allow the use of pumps having smaller displacements. Moreover, since the distance between the process vessel 26 and the filter module 20 will be known and fixed, the dead volume will be a known constant value, and a given pumping efficiency can be guaranteed. This, in turn, enables a pump with a known displacement to be used, and to provide a desired pumping and filtering efficiency, without concern that individual user installations will adversely affect performance.

FIGS. 6 and 7 show non-limiting exemplary drive module arrangements for use with a filter module 20 and filter element. FIG. 6 shows a connection to a diaphragm pump drive module 30 which can be activated by the application of positive and negative pressure. In the illustrated embodiment, the diaphragm pump drive module 30 is coupled to a lower end 21 of the filter module 20. Upper and lower hemispheres 42, 44 of the diaphragm pump drive module 30 are sealed to a bottom surface 45 of the process vessel via at least one circumferential seal 46. An ATF controller 31 is coupled to the lower hemisphere 42 via a positive and negative air pressure supply line for actuating the diaphragm pump drive module 30 to induce ATF of fluid 32 from the process vessel 26 through the filter element (not shown) within the filter module. A fluid harvest port 27 is coupled to tubing 33 and an aseptic coupling 35 disposed in or on the side 37 (or other surface) of the process vessel 26 in a manner previously described to allow permeate to be drawn from the filter module 20 at a desired rate and volume.

FIG. 7 shows a connection to a plunger pump drive module 30 which can be activated by a linear actuator 52. In the illustrated embodiment, the plunger pump drive module 30 is coupled to a lower end 21 of the filter module 20. An upper hemisphere 42 and a plunger 50 of the plunger pump drive module 30 are sealed to a bottom surface 45 of the process vessel 26 via at least one circumferential seal 46. An ATF controller 31 is coupled to the linear actuator 52 via an electrical connection 48. The linear actuator 52 is coupled to the plunger 50 such that movement of the linear actuator moves the plunger 50 toward, or away from, the filter module 20. The ATF controller 31 can thereby control the linear actuator 52 to activate the plunger pump drive module 30 to induce ATF of fluid 32 from the process vessel 26 through the filter element (not shown) within the filter module 20. A fluid harvest port 27 is coupled to tubing 33 and an aseptic coupling 35 disposed in or on the side 37 (or other surface) of the process vessel 26 in a manner previously described to allow permeate to be drawn from the filter module 20 at a desired rate and volume.

Benefits of the disclosed arrangements include reduced time of system deployment, reduced errors in connectivity and potential of contamination, and guaranties consistency in lot to lot runs due to a standard preassembled system design. The resulting simplicity of deployment and use of the proposed one-piece construction of the single-use process vessel and filter system 18 will reduce operator error, will provide consistency of production, and will reduce manufacturing costs.

With advancements in materials and manufacturing methods, a single-use process vessel and filter system 18 of the type described can be set up with minimal handling and does not require cleaning or sterilization by the user. The disclosed system 18 can be supplied sterile and in a form ready to use with minimal preparation and assembly, resulting in cost savings due to reduced labor and handling by the user along with elimination of a long autoclave cycle. Furthermore, at the end of its use, the system 18 can be readily discarded without disassembly or cleaning. The disclosed system 18 reduces risk of contamination and assembly by operators, does not require lengthy validation procedures for operation/sterilization, are lighter and easier to transport, and are less expensive and take up less storage space compared to stainless steel or glass units. In addition, disclosed system 18 eliminates autoclaving which is cumbersome and problematic. Finally, the disclosed system 18 will reduce the amount of liquid waste fluids since they will remove the need for washing parts and parts washing validations. The convenience of the disclosed system 18 will reduce the implementation time, and will minimize or eliminate installation errors, at the manufacturer's site.

In general, the components of the disclosed single-use process vessel and filter system 18 can be constructed from materials that withstand the pressures generated during operation of typical fluid filtration systems, be free of toxins that can harm or kill cells or microorganisms, be readily molded into desired shapes, be light and relatively inexpensive, and must be able to be ethylene oxide (EO) or gamma radiation. For example, useful materials include polycarbonate (PC) (e.g., HPS grade from Sabic), polysulfone (PS), co-poly esters of BPA-free plastics (e.g., TRITAN® from Eastman Chemical Co.), polypropylene (PP), nylon, flexible or elastic materials, glass-filled polymers, ultra-high-molecular-weight polyethylene (UHMWPE), polyether ether ketone (PEEK), and composites (e.g., glass/PC, glass/PS, and glass/nylon). Additional desired features of these materials include their suitability for various manufacturing techniques described herein, amenability to packaging and storage, transportability, biocompatibility, and their protection against damage or contamination of the contents processed therein.

In some non-limiting exemplary embodiments, the filter element is a hollow fiber filter cartridge that comprises multiple hollow fibers that run, in parallel, the length of the cartridge, from a cartridge entrance end to a cartridge exit end. It will be appreciated, however, that the filter element can be any of a variety of other filter types without departing from the spirit of the disclosure. In addition to its use in ATF, the use of variably positioned filter elements can be applied to various filtering processes, such as Tangential Flow Filtration (TFF).

It will be appreciated that the specific filter module, pump module, and process vessel arrangement will depend on the system configuration employed by the user. For example, the arrangements illustrated in FIGS. 2, 4 and 5 show cases where the filter module and pump module are coupled to a tank-type process vessel. In such embodiments, the filter module and pump module may be coupled to the process vessel before a sterilization process (e.g., steam sterilization) is performed. The filter module, pump module and the process vessel are manufactured as independent items, but are assembled together so that the entire system is sterilized together. The arrangements illustrated in FIGS. 3, 6 and 7 show cases in which the filter module, pump module and process vessel can be manufactured, assembled and sterilized at the same time.

In the case of a single use system, the process vessel, filter housing with drive module include on or more aseptic connectors to make appropriate connections therebetween. The process vessel, filter module and drive module may be manufactured and sterilized independently. For cases in which the process vessel, the filter module and the drive module are made of stainless steel, the components can be assembled first and the entire system can be steam sterilized together.

The drive mechanism (e.g., linear actuator or servo where the drive module includes a plunger pump, and a source of compressed gas and vacuum where the drive module includes a diaphragm pump) may not be a permanent part of the drive module. The connection with the plunger or bellows of the drive module will be made via an appropriate coupling.

Where the filter module is made from stainless steel, the diaphragm or plunger of the drive module may be replaceable. In the case of a single use system, the diaphragm or plunger may be a permanent part of the filter module or drive module.

While the present invention has been disclosed with reference to certain embodiments, numerous modifications, alterations and changes to the described embodiments are possible without departing from the spirit and scope of the invention, as defined in the appended claims. Accordingly, it is intended that the present invention not be limited to the described embodiments, but that it has the full scope defined by the language of the following claims, and equivalents thereof.

What is claimed is:

1. A single-use fluid storage and filtration system, comprising:
a process vessel; and
a filter module including a filter element, the filter module fixed to the process vessel;
wherein the filter module is in fluid communication with the process vessel for filtering a fluid received from the process vessel.

2. The single-use fluid storage and filtration system of claim 1, further comprising a drive module coupled to the filter module, the drive module comprising a pump to induce flow of the fluid between the filter module and the process vessel.

3. The single-use fluid storage and filtration system of claim 1, wherein the filter module is disposed within the process vessel.

4. The single-use fluid storage and filtration system of claim 1, wherein the filter module is disposed directly beneath the process vessel.

5. The single-use fluid storage and filtration system of claim 1, further comprising supply and return tubing coupled between the filter module and the process vessel for moving the fluid between the filter module and the process vessel.

6. The single-use fluid storage and filtration system of claim 1, wherein the filter element is a hollow fiber module.

7. The single-use fluid storage and filtration system of claim 1, wherein the process vessel is a bioreactor and the filter element is a hollow fiber module for alternating tangential flow filtration.

8. A single-use fluid storage and filtration system, comprising:
a process vessel;
a filter module including a filter element, the filter module fixed to the process vessel, the filter module in fluid communication with the process vessel for filtering a fluid received from the process vessel; and
a drive module coupled to the filter module, the drive module comprising a pump to induce flow of the fluid between the filter module and the process vessel.

9. The single-use fluid storage and filtration system of claim 8, wherein the filter module is disposed within the process vessel.

10. The single-use fluid storage and filtration system of claim 8, wherein the filter module is disposed directly beneath the process vessel.

11. The single-use fluid storage and filtration system of claim 8, further comprising supply and return tubing coupled between the filter module and the process vessel for moving the fluid between the filter module and the process vessel.

12. The single-use fluid storage and filtration system of claim 8, wherein the filter element is a hollow fiber module.

13. The single-use fluid storage and filtration system of claim 8, wherein the process vessel is a bioreactor and the filter element is a hollow fiber module for alternating tangential flow filtration.

14. A single-use fluid storage and filtration system, comprising:
a bioreactor;
a filter module including a hollow fiber filter element, the filter module fixed to the bioreactor, the filter module in fluid communication with the bioreactor for filtering a fluid received from the bioreactor; and
a drive module coupled to the filter module, the drive module comprising a pump to induce flow of the fluid between the filter module and the bioreactor;
wherein the bioreactor, filter module and drive module comprise a single integrated and sterilized assembly.

15. The single-use fluid storage and filtration system of claim 14, wherein the filter module is disposed within the bioreactor and at least a portion of the drive module is disposed outside of the bioreactor.

16. The single-use fluid storage and filtration system of claim 14, wherein the filter module is disposed directly beneath the bioreactor.

17. The single-use fluid storage and filtration system of claim 14, further comprising supply and return tubing coupled between the filter module and the bioreactor for moving the fluid between the filter module and the bioreactor.
